(19) United States
(12) Patent Application Publication
(10) Pub. No.: US 2025/0009208 A1
(43) Pub. Date: Jan. 9, 2025

(54) BRAIDED SHEATHS FOR MEDICAL DEVICES

(71) Applicant: Boston Scientific Scimed, Inc., Maple Grove, MN (US)

(72) Inventors: Kirsten VIERING, Newton, MA (US); Louis J. BARBATO, Franklin, MA (US)

(73) Assignee: Boston Scientific Scimed, Inc., Maple Grove, MN (US)

(21) Appl. No.: 18/763,129

(22) Filed: Jul. 3, 2024

Related U.S. Application Data
(60) Provisional application No. 63/512,113, filed on Jul. 6, 2023.

(51) Int. Cl. A61B 1/00; A61B 1/018

(52) U.S. Cl. CPC A61B 1/00114; A61B 1/00066; A61B 1/00094; A61B 1/00096; A61B 1/00137; A61B 1/018

(57) ABSTRACT

A medical device includes a shaft and a braided sheath. The braided sheath is coaxial with the shaft and at least extends along the shaft between a distal end of the shaft and a proximal end of the shaft. The braided sheath includes a braiding. The braiding includes a plurality of mechanical strands and at least one conductor configured to deliver energy to or convey signals to and from an electrical device positioned at or near the distal end of the shaft.

### CROSS-REFERENCE TO RELATED APPLICATIONS

This application claims the benefit of priority to U.S. Provisional Application No. 63/512,113, filed on Jul. 6, 2023, which is incorporated by reference herein in its entirety.

### TECHNICAL FIELD

Aspects of the present disclosure generally relate to medical devices and procedures. In particular, aspects of the present disclosure relate to braided sheaths for medical devices

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Endoscopic procedures may be carried out by inserting an insertion device into a patient's body through a surgical incision, or via a natural orifice (e.g., mouth, vagina, or rectum).

The insertion device includes a shaft that includes one or more lumens or working channels therethough. A lumen (or lumens) may often receive various devices and structures such as medical instruments (e.g. irrigation tubes, aspiration tubes, forceps, electrosurgical knives, brushes, RF electrodes, and/or other tools) designed to be operated at a distal end of an insertion device. Surgical incisions and natural orifices are often narrow, and the distal end of the insertion device must be small enough to fit and operate within these spaces. Accordingly, space is limited within the shaft of the distal end, and thus space is also limited in the lumen(s).

The distal end of the insertion device may also include one or more components (lights, cameras, etc.), which require electrical wires or cables in order to be actuated (e.g., powered, controlled, etc.) and/or to be in communication with one or more proximal components (e.g., power sources, controllers, displays, etc.). These electrical wires or cables are ordinarily contained within the shaft of the insertion device. These electrical wires or cables take up space within the shaft, which may limit a size of a lumen within the shaft and/or a number of lumens within the shaft. The electrical wires or cables may also increase a necessary size of the shaft.

The devices and methods of the present disclosure may rectify some of the deficiencies described above or otherwise address other aspects of the art.

### SUMMARY

Each of the aspects disclosed herein may include one or more of the features described in connection with any of the other disclosed aspects. Aspects of the disclosure may related to medical devices with braided sheaths extending along the shafts of said medical devices.

Some of the exemplary devices or methods herein may include any of the following features. A medical device may include a shaft and a braided sheath. The braided sheath may be coaxial with the shaft and may extend at least along the shaft between a distal end of the shaft and a proximal end of the shaft. The braided sheath may include a braiding. The braiding may include: a plurality of mechanical strands and at least one conductor that may be electrically connected and/or communicatively connected to an electrical device positioned at or near the distal end of the shaft.

The medical device may include one or more of the following aspects. The at least one conductor may be a first conductor. The braiding may include a second conductor electrically connected and/or communicatively connected to a second electrical device. The first conductor may be braided clockwise along the braided sheath, and the second conductor may be braided counterclockwise along the braided sheath. The first conductor and second conductor may be both braided clockwise or counter clockwise. The first conductor and second conductor may be electrically connected to an end cap. One of the conductors may be connected to a medical instrument. The medical instrument may be at least partially received by a lumen of the shaft. At least one of the conductors may be a coated core wire. At least one of the conductors may be a stranded wire. At least one of the conductors may be a coaxial cable.

The medical device may include a polyimide wrap layer that covers the braided sheath and extends coaxially with the braided sheath. The polyimide wrap layer may include a first layer and a second layer. The first layer may be a continuous ground plane. The second layer may include one or more conductors. The second layer may include an exterior surface and an interior surface. The one or more conductors of the second layer may be disposed on one of the exterior surface and the interior surface, and a ground layer of the second layer may be disposed on the other of the exterior surface and interior surface. The one or more conductors of the second layer may be impedance controlled conductors.

In another aspect, a medical device may include a shaft. The shaft may include a distal end, a proximal end, and a working channel. The medical device may include a wrap layer coaxial with the shaft and disposed on an outer surface of the shaft. The wrap layer may include one or more conductors.

The medical device may include one or more of the following features. The one or more conductors may be disposed on one of an interior surface of the wrap layer and an exterior surface of the wrap and a ground layer is disposed on the other of the interior surface and the exterior surface. The one or more conductors may be configured to transfer electricity from a handle of the medical device to an electrical device of an end cap at the distal end of the shaft.

In yet another aspect, a medical device may include a handle and a shaft extending from the handle. The shaft may include a proximal end and a distal end. The shaft may include a working channel extending a length of the shaft. The medical device may include an end cap including one or more lights or cameras. The medical device may include a braided sheath coaxial with the shaft and extending along at least an exterior of the shaft between the distal end of the shaft and the proximal end of the shaft. The braided sheath may include a plurality of mechanical strands. The braided sheath may also include a first conductor configured to electrically and/or communicatively connect the handle to one or more lights or cameras in the end cap.

The medical device may include one or more of the following features. The braided sheath may include a second conductor. The first conductor and the second conductor may each include a termination portion positioned at a distal end of the braided sheath. The termination portions may be contained within or electrically connected to a flexible circuit.

### BRIEF DESCRIPTION OF THE FIGURES

The accompanying drawings, which are incorporated in and constitute a part of this specification, illustrate examples of this disclosure and together with the description, serve to explain the principles of the disclosure.

FIG. 1 depicts an exemplary medical device, including an enlarged view of a distal portion of the medical device.

FIG. 2A illustrates a side view of a distal portion of a shaft of the medical device.

FIG. 2B is a lateral cross-sectional view of a portion of the shaft of FIG. 2A.

FIG. 3A depicts a side view of a distal portion of a shaft of the medical device.

FIG. 3B is a lateral cross-sectional view of a portion of the shaft of FIG. 3A.

FIG. 4A shows a side view of a distal portion of a shaft of the medical device.

FIG. 4B is a lateral cross-sectional view of a portion of the shaft of FIG. 4A.

FIG. 5 shows a side view of a portion of another shaft of the medical device.

FIG. 6 shows a side view of a portion of yet another shaft of the medical device.

FIG. 7 shows a side view of a portion of a further shaft of the medical device.

FIG. 8 shows a side view of a portion of another shaft of the medical device.

### DETAILED DESCRIPTION OF THE FIGURES

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device. In contrast, “distal” refers to a position relatively further away from the medical professional using the medical device, or closer to the interior of the body. As used herein, the terms “comprises,” “comprising,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

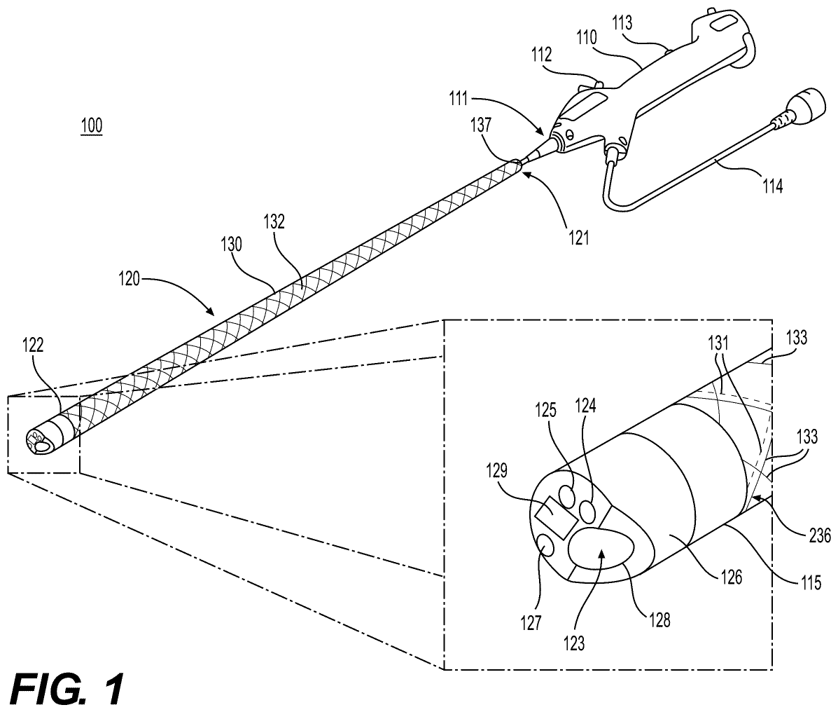

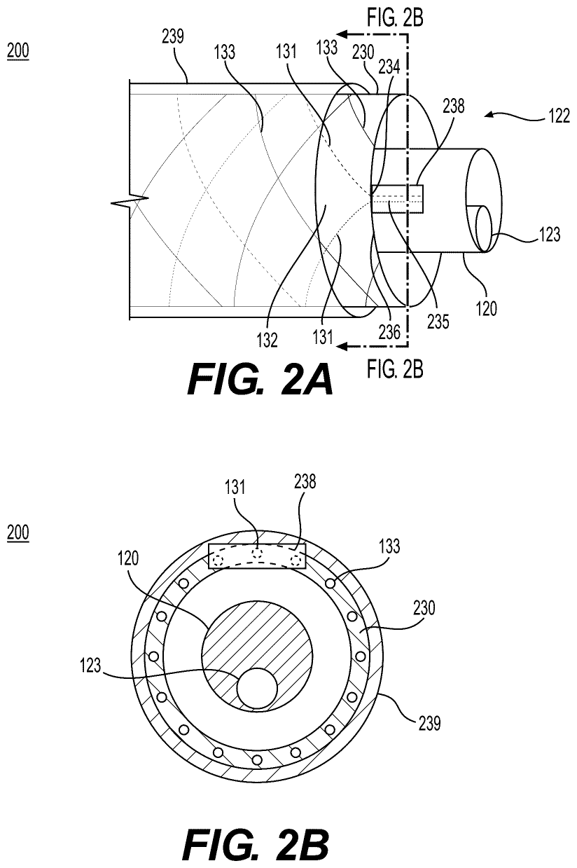

FIG. 1 illustrates aspects of an exemplary medical device 100. Medical device 100 includes a handle 110 and a shaft 120. Shaft 120 may be generally tubular, and may extend from a distal end 111 of handle 110. Shaft 120 includes a proximal end 121 that is positioned at distal end 111 of handle 110 and a distal end 122. Shaft 120 includes a working channel 123 (as seen at least in FIG. 2A) that extends from proximal end 121 of shaft 120 to distal end 122 of shaft 120. In FIGS. 2B, 3B, and 4B, a cross-section of shaft 120 is shown as being a solid, however it should be understood that shaft 120 may be a solid, or hollow, and/or may define multiple working channels, fluid channels and lumens. Medical device 100 may include one or more conductors 131 that electrically connect one or more power sources, controllers, displays, and/or other handle components of handle 110 to appropriate electrical devices positioned at or near a distal end 122 of shaft 120 or within an end cap 126. As discussed in detail below, medical device 100 may include end cap 126 with one or more electrical devices, such as but not limited to one or more of an illumination device or light 127, a visualization device or camera 129. As will further be discussed below, shaft 120 includes a sheath 130 with one or more conductors 131 incorporated into or forming a braiding 132 that at least partially surrounds shaft 120. One or more conductors 131 in braiding 132 electrically or communicatively connect the one or more electrical devices (e.g., light 127, camera 129, etc.) of end cap 126 to handle 110, to other components of medical device 100, or to other devices or systems. One or more conductors 131 may facilitate two way communication, for example, to activate/power camera 129, and also convey video or picture signals proximally from camera 129.

Medical device 100 may receive a portion of a medical instrument (not shown) with an end effector (not shown). Working channel 123 is configured to receive at least a portion of medical instrument. As mentioned, medical device 100 may include end cap 126, for example, at distal end 122 of shaft 120. End cap 126 includes an opening 128, which is in fluid communication with working channel 123 when end cap 126 is coupled to shaft 120. In some aspects, end cap 126 may include one or more fluid channels, 124, 125, which may be fluidly connected to one or more fluid channels (not shown) of shaft 120. For example, fluid channel 124 may be a fluid delivery channel, and fluid channel 125 may be a suction or negative pressure channel.

Although not shown in FIG. 1, sheath 130 may be disposed over or around an outer surface (FIGS. 2-4) of shaft 120. Sheath 130 may be generally tubular and may extend from proximal end 121 of shaft 120 to distal end 122 of shaft 120 or, alternatively, may extend from distal end 111 of handle 110 to distal end 122 of shaft 120. Shaft 120 may include a distal ring portion 115 that may be positioned between end cap 126 and braided sheath 130.

Handle 110 may include one or more ports 112 and one or more valves 113. Port(s) 112 may be positioned on a distal portion of handle 110. Port(s) 112 may be fluidly connected to working channel 123 in shaft 120 and with opening 128 of end cap 126. Port(s) 112 may include a T-connector, a Y-connector, or another appropriate connection. Port(s) 112 may be threaded, may be a Luer component, and/or may include one or more internal flexible seals. Port(s) 112 may be configured to receive one or more instruments (e.g. graspers, guide wires, needles, cautery devices, baskets, etc.) One or more valves 113 may be configured to be actuated to control the delivery of irrigation fluid and/or application of suction, for example, through one or more fluid channels of shaft 120 that are fluidly connected with corresponding fluid channels 124, 125 of end cap 126. Handle 110 may include or otherwise be coupled to one or more power sources, controllers, displays, and other handle components known to one skilled in the art. For example, an umbilicus 114 may extend from handle 110 (e.g., from a distal portion of handle 110) and may carry wires, cables, and/or conduits for providing, for example, power, signals, or fluids to and/or from handle 110. For example, umbilicus 114 may connect handle 110 to one or more user interfaces, monitors, displays, etc.

In one or more embodiments, sheath 130 is a braided sheath. Sheath 130 may extend from distal end 111 of handle 110 to distal end 122 of shaft 120. Sheath 130 may comprise braiding 132 that includes a plurality of elements braided together. Braiding 132 may include a plurality of mechanical strands 133 and one or more conductors 131 (as shown in FIGS. 1 and 2A). The various braidings and one or more conductors, shown in at least FIGS. 1, 2A, 4A, 5, 6, 7 and 8, may include any of the features of the other braidings or one or more conductors, respectively. Plurality of mechanical strands 133 should be understood as being the non-conductive, non-electrical portions of braided sheath 130. For example, plurality of mechanical strands 133 provide shape, support, and/or form to braided sheath 130. Relative to a longitudinal axis of shaft 120, when viewed from proximal end 121 of shaft 120, one or more of plurality of mechanical strands 133 may be braided in a clockwise manner, and one or more of plurality of mechanical strands 133 may be braided in a counter clockwise manner. Plurality of mechanical strands 133 may be configured to provide structure (e.g., rigidity and/or flexibility) to braided sheath 130. Plurality of mechanical strands 133 may also be configured to help prevent the dislocation or movement of one or more members of braiding 132. Braided sheath 130 may be coated and/or otherwise encased in one or more substances or materials (not shown), such as but not limited to plastic, rubber, polymer, silicone, nitrile, neoprene, or other similar substances or materials known in the art. The coating and/or encasing may help to prevent conductor(s) 131 and plurality of mechanical strands 133 from moving during the delivery, positioning, or other usage of medical device 100. In some embodiments, one or more of plurality of mechanical strands 133 may be welded, soldered, or otherwise affixed to one another. For example, two or more of plurality of mechanical strands 133 may be welded, soldered, or otherwise affixed together, which may help to stabilize plurality of mechanical strands 133 and/or braided sheath 130.

It should be understood that the inclusion of one or more conductors 131 within braided sheath 130, instead of within shaft 120, may help to allow for one or more of working channel 123 and opening 128 in end cap 126, and the fluid channels of shaft 120 and corresponding fluid channels 124, 125 of end cap 126 to be greater in size and volume. One or more larger channels may help to allow for larger medical instruments to be received therein, larger volumes of fluid, and for greater suction capabilities. Additionally, the inclusion of one or more conductors 131 within braided sheath 130 may help to allow for one or more additional working channels and fluid channels to be disposed within shaft 120 and/or shaft 120 may be smaller in diameter.

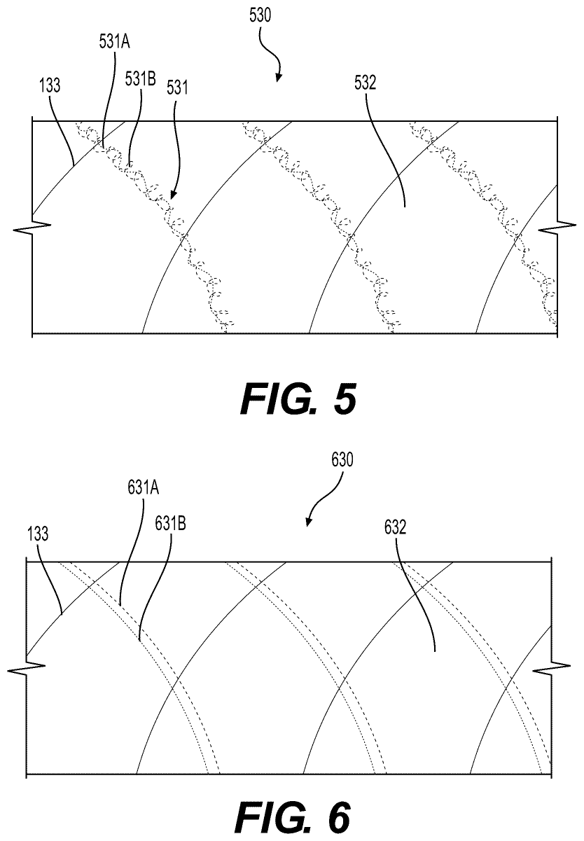

FIGS. 1, 2A-2B, 4A-4B, and 5-8 depict various aspects of exemplary medical devices that include the respective braided sheaths 130, 230, 430, 530, 630, 730, 830. It should be understood that any of braided sheaths 130, 230, 430, 530, 630, 730, 830 may have any of the features of any of the other braided sheaths. As seen in FIGS. 1, 2A, 4A, and 5-8, the various braidings may include one or more conductors 131, 431A, 531, 631A, 631B, 731A, 731B, 831A, 831B. The one or more conductors may be configured to deliver energy at or near distal end 122 of shaft 120. The one or more conductors may be configured to transmit signals to and from the medical instrument or other electrical devices of end cap 126 or at distal end 122. The one or more conductors may be configured to electrically connect and/or communicatively connect components of handle 110 to the medical instrument or electrical devices at or near distal end 122 of shaft 120 or end cap 126. The one or more conductors may be one or more single conductor wires, one or more coated solid core wires, one or more stranded wires, one or more magnet wires, etc. In some aspects, the one or more conductors may be one or more cable assemblies, such as but not limited to one or more coaxial cables, one or more shielded twin axial cables, and one or more ribbon cable assemblies. The one or more conductors may replace one or more of plurality of mechanical strands 133, or may be additionally interwoven with plurality of mechanical strands 133. Relative to shaft 120, the one or more conductors may be woven in a clockwise or counter clockwise manner. In some embodiments, relative to a longitudinal axis of shaft 120 when viewed from proximal end 121 of shaft 120, one of the one or more conductors may be woven in a clockwise manner and another one of the one or more conductors may be woven in a counter clockwise manner. It should be understood that incorporating any number of conductors into braided sheath in a clockwise and/or counter clockwise manner is contemplated within the scope of this disclosure. In embodiments that include at least one of the one or more conductors travelling in a clockwise manner and another at least one of the one or more conductors travelling in a counter clockwise manner (see FIG. 5C), this may increase the number of instances that the two conductors cross over each other across a length of the braided sheath. Nevertheless, the opposite winding arrangement may help to decrease instances of parallelism of the respective conductors. Limiting parallelism of the conductors may help to reduce cross-talk between conductors. Cross-talk is defined as any phenomenon by which a signal transmitted on one circuit or channel of a transmission system creates an undesired effect in another circuit or channel. Cross-talk is usually caused by undesired capacitive, inductive, or conductive coupling from one circuit or channel to another. In some embodiments, the braided sheath may be configured to protect against unwanted electromagnetic transmissions traveling to and/or traveling from shaft 120.

FIGS. 2A and 2B depict aspects of an exemplary medical device 200. For example, FIG. 2A illustrates a distal portion of shaft 120, and FIG. 2B illustrates a lateral cross-sectional view of a portion of shaft 120. Although not shown, a distal portion of shaft 120 may be coupled to an end cap (e.g., end cap 126), as discussed above. Shaft 120 includes a braided sheath 230 that is coaxial with shaft 120. Shaft 120 may further include an exterior insulation layer 239 covering (e.g., radially surrounding) braided sheath 230. Braided sheath 230 may have the same of or all of the same features as braided sheath 230 as described above. Exterior insulation layer 239 may help to limit or prevent the transfer of electricity from braided sheath 230 and/or shaft 120, for example, to the environment (e.g., the patient's body). Exterior insulation layer 239 may be further configured to limit or prevent electromagnetic inference from the environment to braided sheath 230 and/or shaft 120. Exterior insulation layer 239 may extend from proximal end 121 of shaft 120 to distal end 122 of shaft 120. One or more conductors 131 of braided sheath 230 may include one or more distal termination portion(s) 235 that extend(s) beyond a distal termination 236 of plurality of mechanical strands 133. Braided sheath 230 may include a flexible circuit 238 that extends beyond distal termination 236. Distal termination portion 235 may be contained in or otherwise electrically connected to flexible circuit 238. In some embodiments, one or more conductors 131 may be connected to one or more copper traces (not shown) within flexible circuit 238. In one or more embodiments, flexible circuit 238 may be coupled to (e.g., plugged into) a compatible port (not shown) of an electrical circuit, or circuit board, and electrically connected to the electrical device(s) of end cap 126 or otherwise positioned at distal end 122 of shaft 120. In some embodiments, shaft 120 may include a layer of insulation (not shown) between an outer surface of shaft 120 and braided sheath 230.

As shown in FIG. 2A, one or more conductors 131 may include a distal termination portion 235. One or more conductors 131 may converge into one distal termination portion 235, or may each include a respective distal termination portion 235. Distal termination portion 235 is a portion of conductor(s) 131 that extends distally beyond a distal termination 236 of plurality of mechanical strands 133. In some embodiments, distal termination portion 235 of conductor(s) 131 may be directly connected to electrical devices positioned at or near distal end 122 of shaft 120. In one or more embodiments, distal termination portion(s) 235 may be electrically connected to electrical circuits (not shown) or a circuit board (not shown) of end cap 126 and by extension the electrical devices of end cap 126. The electrical circuits or circuit board of end cap 126 may be printed, 3-D printed, chemically etched, implemented by laser direct structuring, or otherwise formed onto or as a proximal facing surface of end cap 126 or any other surface of end cap 126. In these aspects, the electrical circuits or circuit board of end cap 126 may be internal to end cap 126, for example, electrically connected to one or more of light 127, camera 129, etc.

As seen in FIG. 2A, plurality of mechanical strands 133 and one or more conductors 131 may be configured so that two or more of conductors 131 intersect, or otherwise overlap, at predetermined points along a length of braided sheath 230. Braided sheath 230 may be sized so that distal termination 236 of plurality of mechanical strands 133 is positioned at an intersection or convergence 234 of two or more of conductors 131. This configuration may help to allow for distal termination portion(s) 235 of the two or more conductors 131 to be more easily contained or connected to flexible circuit 238 and end cap 126, and may further minimize a length of a circuit between the distal termination 236 and an electrical device positioned at or near distal end 122 of shaft 120. Minimizing the lengths of circuits between distal termination 236 and electrical devices may prevent the crowding of electrical wires at or near distal end 122 of shaft 120.

In one or more embodiments, distal termination portion 235 may be contained in or electrically connected to flexible circuit 238. In some aspects, flexible circuit 238 may be a card-edge type flat flexible circuit that may be electrically connected to one or more electrical devices or alternatively may be electrically connected to end cap 126 as described above. Flexible circuit 238 may extend distally from distal termination 236 of plurality of mechanical strands 133. In some embodiments, distal termination portion 235 may be electrically connected to one or more flex boards (not shown) that contain or are electrically connected to one or more electrical devices positioned at or near distal end 122 of shaft 120 or electrically connected to end cap 126, as described above. The one or more flex boards may be routed as needed within end cap 126 to place the one or more electrical devices in desirable locations.

One or more conductors 131 may include a proximal termination portion (not shown). One or more conductors 131 may converge into one proximal termination portion or each include their own proximal termination portion. The proximal termination portion is a portion of conductor(s) 131 that extends proximally beyond a proximal termination 137 (FIG. 1) of plurality of mechanical strands 133. The proximal termination portion may be directly connected to handle components of handle 110, for example, such as but not limited to power sources, controllers, displays, and other handle components known to one skilled in the art. In some embodiments, the proximal termination portion may be electrically connected to one or more flex boards (not shown) that contain or are electrically connected to handle components of handle 110. In one or more embodiments, the proximal termination portion may be contained in or electrically connected to a flexible circuit (not shown), for example, a card-edge type flat flexible circuit, that may be electrically connected to the handle components of handle 110. In some embodiments, one or more conductors 131 may be connected to one or more copper traces within the flexible circuit. The flexible circuit may extend proximally of proximal termination 137 of plurality of mechanical strands 133. One or more conductors 131 may be electrically and/or communicatively coupled to one or more elements in umbilicus 114, for example, to connect to one or more power sources, controllers, displays, etc.

It should be understood that the one or more conductors of the various braidings shown in FIGS. 1, 4, 5, 6, 7, 8 may include similar distal termination portions and distal connections and proximal termination portions and proximal connections as described in the preceding paragraphs.

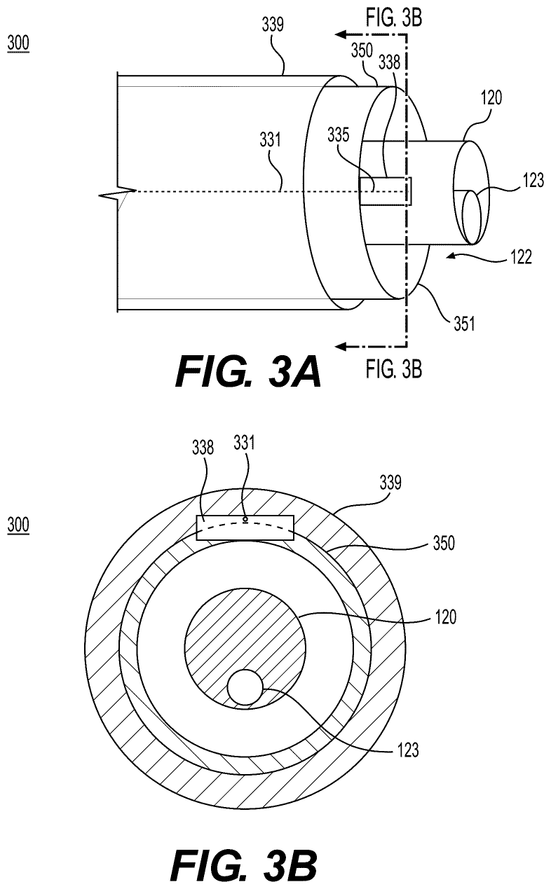

FIGS. 3A and 3B illustrate aspects of another exemplary medical device 300. Shaft 120 includes a wrap layer 350 that is coaxial with shaft 120. Wrap layer 350 may be used to create an electrical conduit, such as but not limited to an electrical ground. Wrap layer 350 may be used in addition to braided sheath 230. Wrap layer 350 may be used for transmitting high-speed differentials signals that require inter and intra pair matching. Alternatively or additionally, wrap layer 350 may be used for impedance control. Wrap layer 350 may extend from proximal end 121 (FIG. 1) of shaft 120 to distal end 122 of shaft 120. Shaft 120 may further include an insulation layer 339 covering (e.g., radially surrounding) wrap layer 350. Insulation layer 339 may extend from proximal end 121 (FIG. 1) of shaft 120 to distal end 122 of shaft 120. Wrap layer 350 may contain one or more conductors 331. One or more conductors 331 may be one or more single conductor wires, one or more coated solid core wires, one or more stranded wires, one or more magnet wires, etc. In some aspects, one or more conductors 331 may be one or more cable assemblies, such as but not limited to one or more coaxial cables, one or more shielded twin axial cables, and one or more ribbon cable assemblies. One or more conductors 331 may have any of the features of 131, 431A, 431B, 531, 631A, 631B, 731A, 731B, 831A, 831B. In some embodiments, shaft 120 may include a layer of insulation (not shown) between an outer surface of shaft 120 and wrap layer 350.

Wrap layer 350 may include a ground layer. The ground layer may be disposed on one of an interior surface and an exterior surface of wrap layer 350, and one or more conductors 331 may be disposed on the other of the interior surface and exterior surface of wrap layer 350. In some embodiments, one or more conductors 331 may be two or more matched-length conductors configured to transmit high-speed signals. It should be understood that for certain high-speed signals, length matching is necessary. As shown in FIG. 3A, one or more conductors 331 may extend linearly along a length of wrap layer 350. However, one or more conductors 331 are not limited to extending linearly along wrap layer 350. Alternatively, one or more conductors 331 may wind around a surface of wrap layer 350, for example, in a clockwise or counterclockwise manner similarly to the one or more conductors of the various braided sheaths disclosed in this disclosure.

One or more conductors 331 of wrap layer 350 may electrically connect and/or communicatively connect the handle components of handle 110 with the electrical devices positioned at or near distal end 122 of shaft 120 or end cap 126. Wrap layer 350 may also help to limit electromagnetic emissions and electromagnetic inference to and from shaft 120. In some embodiments, conductor(s) 331 include an electrical ground. One or more conductors 331 of wrap layer 350 may be electrically connected to a medical instrument at least partially received within a working channel 123 of shaft 120. Conductor(s) 331 of wrap layer 350 may help to facilitate electrical connection and/or communication between the handle components within handle 110 and the medical instrument at least partially received within working channel 123.

A distal termination portion 335 of one or more conductors 331 may extend beyond a distal termination 351 of wrap layer 350 may extend into a flexible circuit 338. In some embodiments, one or more conductors 331 may be connected to one or more copper traces (not shown) within flexible circuit 338. In some embodiments, distal termination portion 335 of one or more conductors 331 may be directly connected to electrical devices positioned at or near distal end 122 of shaft 120. In one or more embodiments, distal termination portion 335 may be electrically connected to electrical circuits (not shown) or a circuit board (not shown) of end cap 126 (FIG. 1), and the electrical circuits or the circuit board may then be electrically connected to the electrical devices of end cap 126 (FIG. 1).

The electrical circuits or circuit board of end cap 126 may be printed, 3-D printed, chemically etched, implemented by laser direct structuring onto a proximal facing surface of end cap 126 or any other surface of end cap 126. In one or more embodiments, distal termination portion 335 may be contained in or electrically connected to flexible circuit 338. In some aspects, flexible circuit 338 may be a card-edge type flat flexible circuit that may be electrically connected to one or more electrical devices or, alternatively, may be electrically connected to end cap 126, as described above. Flexible circuit 338 may extend distally from distal termination 351 of wrap layer 350. In some embodiments, distal termination portion 335 may be electrically connected to one or more flex boards (not shown) that contain or are electrically connected to one or more electrical devices positioned at or near distal end 122 of shaft 120 or electrically connected to end cap 126, as described above. The one or more flex boards may be routed as needed within end cap 126 to place the one or more electrical devices in desirable locations.

One or more conductors 331 of wrap layer 350 may include a proximal termination portion (not shown). One or more conductors 331 may converge into one proximal termination portion or each include their own proximal termination portion. The proximal termination portion is a portion of the one or more conductors 331 that extends proximally beyond a proximal termination (not shown) of wrap layer 350. The proximal termination portion may be directly connected to components of handle 110, for example, such as but not limited to power sources, controllers, displays, and other handle components known to one skilled in the art. In some embodiments, the proximal termination portion may be electrically connected to one or more flex boards (not shown) that contain or are electrically connected to components of handle 110. In one or more embodiments, the proximal termination portion may be contained in or electrically connected to a flexible circuit (not shown), for example, a card-edge type flat flexible circuit, that may be electrically connected to the handle components of handle 110. In some embodiments, one or more conductors 331 may be connected to one or more copper traces within the flexible circuit. The flexible circuit may extend proximally of the proximal termination of wrap layer 350. One or more conductors 331 may be electrically and/or communicatively coupled to one or more elements in umbilicus 114, for example, to connect to one or more power sources, controllers, displays, etc.

It should be understood that the inclusion of one or more conductors 331 within wrap layer 350 instead of within shaft 120 may help to allow for one or more of working channel 123 and opening 128 in end cap 126, and the fluid channels of shaft 120 and corresponding fluid channels 124, 125 of end cap 126 to be greater in size (e.g., in cross-sectional area). One or more larger channels may help to allow for larger medical instruments to be received therein, larger volumes of fluid, and for greater suction capabilities. Additionally, the inclusion of one or more conductors 331 within wrap layer 350 may help to allow for one or more additional working channels and fluid channels to be disposed within shaft 120 and/or for shaft 120 to be smaller in diameter.

In some embodiments, any of the braided sheaths 130, 230, 430, 530, 630, 730, 830 may be disposed over wrap layer 350 and extend from proximal end 121 of shaft 120 to distal end 122 of shaft 120. At distal end 122 of shaft 120, the conductors of the braided sheath and wrap layer 350 may distally extend into flexible circuit 338, one or more flex boards, or be electrically connected to end cap 126 or a medical instrument. The one or more flex boards may be routed as needed within end cap 126 to place the one or more electrical devices in desirable locations. At proximal end 121 of shaft 120, the conductors of the braided sheath and wrap layer 350 may proximally extend into a flexible circuit, one or more flex boards, or be electrically connected to components of handle 110.

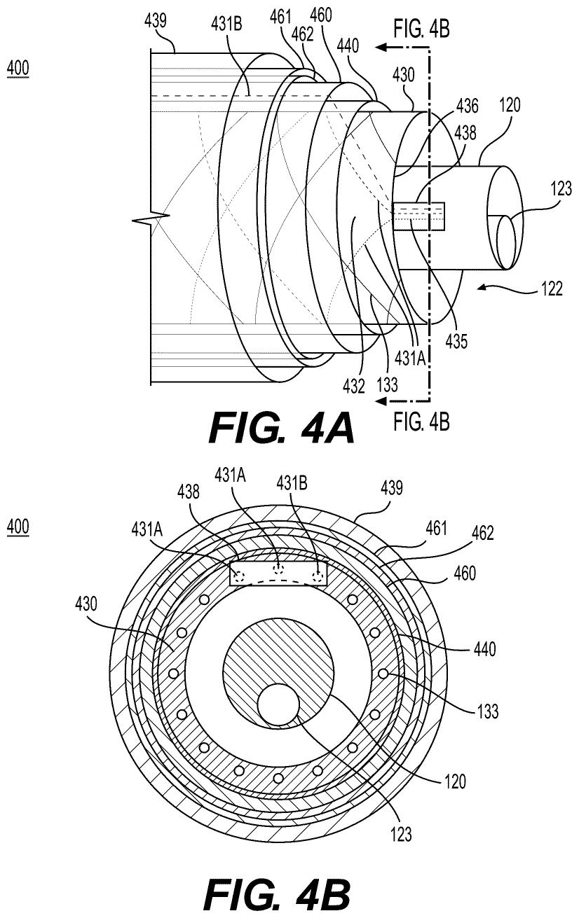

FIGS. 4A and 4B depict aspects of yet another exemplary medical device 400 that combines aspects of exemplary medical device 200 disclosed in FIGS. 2A and 2B with aspects of medical device 300 disclosed in FIGS. 3A and 3B. Shaft 120 includes a braided sheath 430. Braiding 432 of braided sheath 430 may include a plurality of mechanical strands 133 and one or more conductors 431A. A distal termination portion 435 of one or more conductors 431A may extend into a flexible circuit 438. Braided sheath 430 may have some of or all of the same features as braided sheath 230 and/or braided sheath 130 as described above. Shaft 120 further includes an interior layer of insulation 440 covering (e.g., radially surrounding) braided sheath 430. Interior layer of insulation 440 may extend from proximal end 121 (FIG. 1) of shaft 120 to distal end 122 of shaft 120. Shaft 120 further includes a first polyimide layer 460 covering (e.g., radially surrounding) interior layer of insulation 440. Shaft 120 also include a second polyimide layer 461 covering (e.g., radially surrounding) first polyimide layer 460. First polyimide layer 460 and second polyimide layer 461 may extend from proximal end 121 (FIG. 1) of shaft 120 to distal end 122 of shaft 120. Within this disclosure, first polyimide layer 460 and second polyimide layer 461 may be referred to collectively as the polyimide wrap layer, that is, the polyimide wrap layer may include or comprise a plurality of layers. The polyimide wrap layer may be used for transmitting high-speed differentials signals that require inter and intra pair matching. Shaft 120 further includes an exterior layer of insulation 439 that covers (e.g., radially surrounds) second polyimide layer 461. Shaft 120 may include an intermediate layer of insulation 462 between first polyimide layer 460 and second polyimide layer 461. It should be understood that shaft 120 may include additional layers of insulation exterior to second polyimide layer 461 and/or interior to first polyimide layer 460, in addition to those insulation layers already discussed. In some embodiments, shaft 120 may include a layer of insulation (not shown) between an outer surface of shaft 120 and braided sheath 430.

In one or more embodiments, one of first polyimide layer 460 or second polyimide layer 461 may include one or more conductors 431B and the other of the first polyimide layer 460 and second polyimide layer 461 may be a ground layer. The polyimide layer with the one or more conductors 431B may include a ground layer. The ground layer may be disposed on one of an interior surface of the polyimide layer and an exterior surface of the polyimide layer and the one or more conductors 431B are disposed on the other of the interior surface of the polyimide layer and the exterior surface of the polyimide layer.

As shown in FIG. 4A, one or more conductors 431A, 431B of braided sheath 430 and one of polyimide layers 460, 461 may include a distal termination portion 435. One or more conductors 431A, 431B may converge into one distal termination portion 435, or may each include a respective distal termination portion 435. Distal termination portion 435 includes a portion of respective conductor(s) 431A, 431B that extends distally beyond a distal termination 436 of plurality of mechanical strands 133. In some embodiments, distal termination portion 435 of conductor(s) 431A, 431B may be directly connected to electrical devices positioned at or near distal end 122 of shaft 120. In one or more embodiments, distal termination portion(s) 435 may be electrically connected to electrical circuits (not shown) or a circuit board (not shown) of end cap 126 (FIG. 1), and the electrical circuits or the circuit board may then be electrically connected to the electrical devices of end cap 126 (FIG. 1). The electrical circuits or circuit board of end cap 126 may be printed, 3-D printed, chemically etched, implemented by laser direct structuring onto a proximal facing surface of end cap 126 or any other surface of end cap 126. In one or more embodiments, distal termination portion 435 may be contained in or electrically connected to a flexible circuit 438. In some embodiments, one or more conductors 431A, 431B may be connected to one or more copper traces (not shown) within flexible circuit 438. In some aspects, flexible circuit 438 may be a card-edge type flat flexible circuit that may be electrically connected to one or more electrical devices or alternatively may be electrically connected to end cap 126 as described above. Flexible circuit 438 may extend distally from distal termination 436 of plurality of mechanical strands 133. In some embodiments, distal termination portion 435 may be electrically connected to one or more flex boards (not shown) that contain or are electrically connected to one or more electrical devices positioned at or near distal end 122 of shaft 120 or electrically connected to end cap 126, as described above. The one or more flex boards may be routed as needed within end cap 126 to place the one or more electrical devices in desirable locations.

As mentioned above, one or more conductors 431A, 431B of braided sheath 430 and one of polyimide layers 460, 461 may include a proximal termination portion (not shown). One or more conductors 431A, 431B may converge into one proximal termination portion or each include their own proximal termination portion. The proximal termination portion is a portion of conductor(s) 431A, 431B that extends proximally beyond a proximal termination 137 (FIG. 1) of plurality of mechanical strands 133. The proximal termination portion may be directly connected to components of handle 110, for example, such as but not limited to power sources, controllers, displays, and other handle components known to one skilled in the art. In some embodiments, the proximal termination portion may be electrically connected to one or more flex boards (not shown) that contain or are electrically connected to handle components of handle 110. In one or more embodiments, the proximal termination portion may be contained in or electrically connected to a flexible circuit (not shown), for example, a card-edge type flat flexible circuit, that may be electrically connected to the components of handle 110. In some embodiments, one or more conductors 431A, 431B may be connected to one or more copper traces within the flexible circuit. The flexible circuit may extend proximally of proximal termination 137 of plurality of mechanical strands 133. The one or more conductors 431A, 431B may be electrically and/or communicatively coupled to one or more elements in the umbilicus 114, for example, to connect to one or more power sources, controllers, displays, etc.

The above-described layered arrangement of braided sheath 430, interior layer of insulation 440, first polyimide layer 460, intermediate layer of insulation 462, second polyimide layer 461, and exterior layer of insulation 439 may help to provide numerous advantages. For example, the layered arrangement may help to facilitate the segregation of conductors that require impedance control and conductors that do not require impedance control. Conductor(s) (e.g. one or more conductors 431B) that do require impedance control may be placed in either first polyimide layer 460 or second polyimide layer 461, with the remaining polyimide layer not containing the conductor(s) requiring impedance control may be a continuous ground plane. Conductor(s) (e.g. one or more conductors 431A) that do not require impedance control may be interwoven within braided sheath 430. The segregation may be necessary for conductors that require impedance control. First polyimide layer 460 and second polyimide layer 461 are two conducting layers, one for the impedance controlled signal conductors and the other layer for a ground plane. The ground plane distance to one or more conductors 431B and the dielectric constant of the polyimide contributes to the impedance of one or more conductors 431B. The non-impedance controlled conductors (e.g., one or more conductors 431A) may be interwoven in braiding 432 because a solid ground reference is not necessary. First polyimide layer 460 and second polyimide layer 461 may be thin and flexible, for example, so as to have minimal mechanical impact on shaft 120. It should be understood that one or more conductors 431A, 431B may be any of the features of one or more conductors 131, 331, 531, 631A, 631B, 731A, 731B, 831A, 831B.

In some embodiments, one or more conductors 431B may be two or more matched-length conductors configured to transmit high-speed signals. It should be understood that for certain high-speed signals, length matching may be necessary. As shown in FIG. 4A, one or more conductors 431B may travel linearly along the polyimide layer (for example within first polyimide layer 460). However, one or more conductors 431B are not limited to traveling linearly along or within the polyimide layer, but instead may wind around on a surface of or within the polyimide layer in a clockwise or counterclockwise manner, for example, in a similar manner as the one or more conductors of the various braided sheaths disclosed in this disclosure.

FIG. 5 illustrates an aspect of an exemplary medical device that includes a braided sheath 530. Braided sheath 530 includes at least one conductor 531. Conductor 531 may include two conductors 531A, 531B intertwined together. Conductors 531A, 531B may be braided clockwise or counter clockwise about a braiding 532. Conductor 531 may be a twisted pair of conductors.

Another aspect of an exemplary medical device is shown in FIG. 6. The medical device includes a braided sheath 630. Braided sheath 630 may include two conductors 631A, 631B. The two conductors 631A, 631B may be a twin axial cable. Conductors 631A, 631B intertwined together and may be braided clockwise or counter clockwise about a braiding 632.

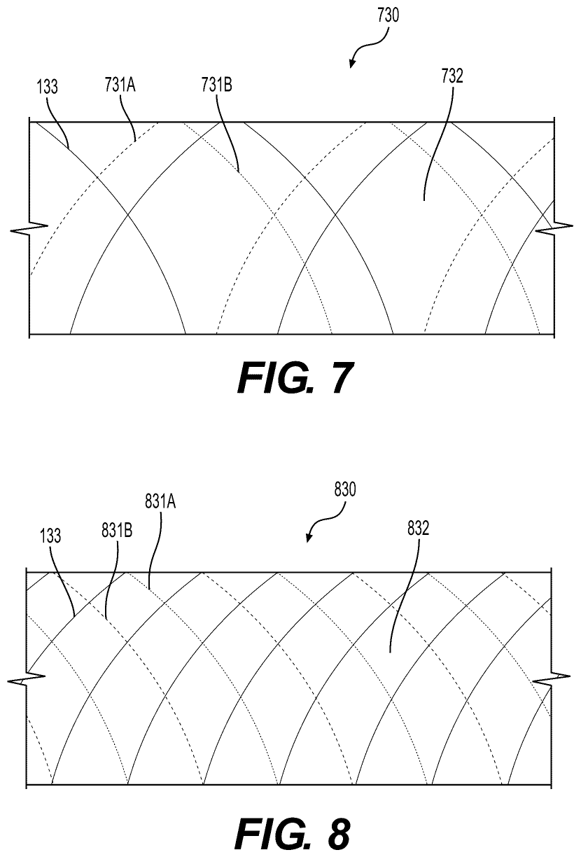

An aspect of an exemplary medical device is shown in FIG. 7. The medical device includes a braided sheath 730. Braided sheath 730 may include a first conductor 731A and a second conductor 731B. The two conductors 731A, 731A may be interwoven with a braiding 732. First conductor 731A may be braided clockwise, and second conductor 731B may be braided counter clockwise. Relative to a longitudinal axis of shaft 120, when viewed from proximal end 121 of shaft 120, arranging one of conductors 731A, 731B to travel clockwise about braiding 732 and the other of conductors 731A, 731B to travel counter clockwise about braiding 732 may help to limit instances of parallelism between the two conductors 731A, 731B, while conductors 731A, 731B extend along braided sheath 730.

FIG. 8 illustrates another aspect of an exemplary medical device. The medical device includes a braided sheath 830. Braided sheath 830 may include two conductors 831A, 831B. The two conductors 831A, 831B may be interwoven with braiding 832 and both extend in either clockwise or counter-clockwise. The conductors 831A, 831B may be incorporated into braidings by existing braiding machines by replacing one or more non-conductive strands with one or more conductors.

An aspect of the present disclosure is a method of manufacturing braided sheath with one or more conductors, as described in various aspects above. Braided sheaths known in the art may be formed by providing a plurality of spools of mechanical strands, the spools of mechanical strands each including a leading end, arranging the spools of mechanical strands so that leading ends of the spools of mechanical strands may be fed into a receiving end of a braiding machine, and actuating the braiding machine to form the braided sheath. The braiding machine may be a horn gear braiding machine. A braided sheath may be formed by providing a plurality of spools of mechanical strands, providing one or more spools of conductor, wherein the spools of the mechanical strands and spools of conductors each include a leading end, arranging the spools of mechanical strands and spools of conductors so that the leading ends of the spools may be fed into a receiving end of a braiding machine, actuating the braiding machine to for braided sheath. The method of manufacturing the braided sheath may further comprise arranging one or more spools of conductor to be wound counter-clockwise relative to a longitudinal axis defined by a forming braided sheath and arranging one or more spools of conductor to be wound clockwise relative to the longitudinal axis defined by the forming braided sheath. In one or more embodiments, the spools of conductors may be formed of one or more single conductor wires, one or more coated solid core wires, one or more stranded wires, etc. Alternatively or additionally, the spools of conductors may include one or more cable assemblies, such as, but not limited to, one or more coaxial cables and one or more shielded twin axial cables, and combinations thereof.

As discussed above, removing the electrical wires or cables from within the shaft and disposing the electrical wires or cables in the various braided sheaths, wrap layers and polyimide wrap layers disclosed herein may help to allow for larger lumens for larger instruments, more lumens for more instruments, more lumens for delivery of suction and different types of fluid, larger lumen(s) for delivery of greater amounts of fluid(s), larger lumen(s) for application of suction, smaller shaft/insertion portion diameters, etc. Further, the braided sheaths disclosed above may help to reduce parallelism of conductors by braiding one or more conductors clockwise and one or more other conductors counter clock-wise. Additionally, as disclosed in FIGS. 4A and 4B, the present disclosure may help to facilitate the segregation of impedance controlled conductors and non-impedance controlled conductors. One skilled in the art would recognize the above described benefits as significant advancements in the art.

Although, the braided sheaths, polyimide wrap layers, insulation layers, and wrap layer are shown in FIGS. 1-8 and discussed in this disclosure as covering an external shaft (for example, shaft 120), the braided sheaths, polyimide wrap layers, insulation layers, and wrap layer may be applied to internal shafts (for example, working channel 123) in the same or similar manners as discussed above with respect to shaft 120.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A medical device comprising:
a shaft; and
a braided sheath coaxial with the shaft and at least extending along the shaft between a distal end of the shaft and a proximal end of the shaft;
wherein the braided sheath includes a braiding, and wherein the braiding includes:
a plurality of mechanical strands, and
at least one conductor electrically connected and/or communicatively connected to a first electrical device positioned at or near the distal end of the shaft.

2. The medical device of claim 1, wherein the at least one conductor is a first conductor, wherein the braiding includes a second conductor electrically connected and/or communicatively connected to a second electrical device.

3. The medical device of claim 2, wherein the first conductor is braided clockwise along the braided sheath and the second conductor is braided counterclockwise along the braided sheath.

4. The medical device of claim 2, wherein the first conductor and the second conductor are both either braided clockwise or counterclockwise.

5. The medical device of claim 2, wherein the first conductor and the second conductor are electrically connected to an end cap.

6. The medical device of claim 2, wherein the first conductor is connected to a medical instrument, wherein the medical instrument is at least partially received by a lumen of the shaft.

7. The medical device of claim 1, wherein the at least one conductor is a coated core wire.

8. The medical device of claim 1, wherein the at least one conductor is a stranded wire.

9. The medical device of claim 1, wherein the at least one conductor is a coaxial cable.

10. The medical device of claim 1, further comprising a polyimide wrap layer that covers the braided sheath and extends coaxially with the braided sheath.

11. The medical device of claim 10, wherein the polyimide wrap layer includes a first layer and a second layer.

12. The medical device of claim 11, wherein the first layer is a continuous ground plane.

13. The medical device of claim 12, wherein the second layer includes one or more conductors.

14. The medical device of claim 13, wherein the second layer includes an exterior surface and an interior surface, wherein the one or more conductors of the second layer are disposed on one of the exterior surface and the interior surface, and a ground layer of the second layer is disposed on the other of the exterior surface and the interior surface.

15. The medical device of claim 13, wherein the one or more conductors are impedance controlled conductors.

16. A medical device comprising:
a shaft including a distal end, a proximal end, and a working channel; and
a wrap layer, wherein the wrap layer is coaxial with the shaft and disposed on an outer surface of the shaft, and wherein the wrap layer includes one or more conductors.

17. The medical device of claim 16, wherein the one or more conductors are disposed on one of an interior surface of the wrap layer and an exterior surface of the wrap layer and a ground layer is disposed on the other of the interior surface and the exterior surface.

18. The medical device of claim 16, wherein the one or more conductors are configured to transfer electricity from a handle of the medical device to an electrical device of an end cap at the distal end of the shaft.

19. A medical device comprising:
a handle;
a shaft extending from the handle, wherein the shaft includes a proximal end and a distal end, wherein the shaft includes a working channel extending a length of the shaft;
an end cap include one or more lights or cameras; and
a braided sheath coaxial with the shaft and extending along at least an exterior of the shaft between the distal end of the shaft and the proximal end of the shaft;
wherein the braided sheath includes:
a plurality of mechanical strands; and
a first conductor configured to electrically and/or communicatively connect the handle to the one or more lights or cameras in the end cap.

20. The medical device of claim 19, wherein the braided sheath includes a second conductor, wherein the first conductor and the second conductor each include a termination portion positioned at a distal end of the braided sheath; wherein the termination portions are contained within or electrically connected to a flexible circuit.
